(19) United States
(12) Patent Application Publication
(10) Pub. No.: US 2024/0218006 A1
(43) Pub. Date: Jul. 4, 2024

(54) CONCENTRATION AND DIAFILTRATION OF OLIGONUCLEOTIDES

(71) Applicant: Hydranautics, Oceanside, CA (US)

(72) Inventors: Anand Kumar KRISHNAMURTHY, Worcester, MA (US); Taisuke YAMAGUCHI, Kusatsu (JP); Karsten KELLER, Mendon, MA (US); Craig R. BARTELS, San Diego, CA (US)

(21) Appl. No.: 18/288,754

(22) PCT Filed: Dec. 16, 2021
(86) PCT No.: PCT/US21/63759

§ 371 Date: Oct. 27, 2023

Related U.S. Application Data
(60) Provisional application No. 63/182,147, filed on Apr. 30, 2021.

(51) Int. Cl. C07H 1/06; B01D 61/02; B01D 61/14; B01D 71/68

(52) U.S. Cl. CPC C07H 1/06; B01D 61/027; B01D 61/145; B01D 71/68; B01D 2315/10; B01D 2315/16; B01D 2325/14; B01D 2325/20

(57) ABSTRACT

A method for concentration of oligonucleotides from a solution comprising negatively charged oligonucleotides is provided. The method includes the steps of: circulating the solution through an ultrafiltration or nanofiltration unit having a membrane, filtering the solution through the membrane to remove salts from the solution and obtain a retentate solution including the oligonucleotides and a permeate solution including the removed salts, diafiltering the retentate solution with a diafiltration buffer to produce a concentrated oligonucleotide solution, and collecting the concentrated oligonucleotide solution. The membrane has a nominal molecular weight cutoff in the range of from about 700 to about 5000 daltons, a negatively charged surface with a zeta potential of −20 mV or lower, and a water flux in the range of 800 to 1500 ml/min/m².

### CROSS-REFERENCE TO RELATED APPLICATIONS

This application claims priority to U.S. Provisional Patent Application No. 63/182,147, filed Apr. 30, 2021, the entire disclosure of which is herein incorporated by reference.

### BACKGROUND OF THE INVENTION

The present invention relates to concentration and diafiltration of oligonucleotides, and more particularly of negatively charged oligonucleotides.

Oligonucleotide molecules are short single strands of synthetic DNA or RNA. An oligonucleotide is a macromolecule comprising a sequence of nucleosides, each of which includes a sugar and a nucleobase. Each nucleoside is separated from adjacent nucleosides with an internucleosidic linkage, which effectively serves to bond the nucleosides together. The sugar can be a pentose, such as a deoxyribose, ribose, or 2′-0-substituted ribose. A number of different bases and substituted bases can be used, the four most common of which are adenine, cytosine, guanine, and thymine (abbreviated as A, C, G, and T, respectively). The internucleosidic linkage is most commonly a phosphate, which may be substituted with a variety of substituents at a nonbridging oxygen atom, most commonly by sulphur or an alkyl, ester, or amide group.

Oligonucleotides have shown significant promise for many molecular biology and synthetic biology applications, such as genetic testing and forensic research. Additionally, oligonucleotides are used to control gene expression in living cells and have demonstrated significant therapeutic effects for rare and common disorders. There are multiple oligonucleotide-based therapeutic drugs in the clinical stage, and some of these drugs have being approved for commercial use. There is, therefore, a great need for the large-scale production of oligonucleotides for commercial application, particularly due to their specificity for complementary nucleotide sequences in DNA or RNA obtained from biological samples. Referring to FIG. 1A, there is shown a schematic diagram of the manufacturing process for RNA oligonucleotides, and referring to FIG. 1B, there is shown a schematic diagram of the manufacturing process for DNA oligonucleotides.

Different methods are used for synthesizing oligonucleotides, including phosphoramidite, phosphotriester, and H-phosphonate methods, each of which is generally known in the field of biochemistry. The predominant manufacturing process for oligonucleotide synthesis is the phosphoramidite method. Briefly, generally, for synthesis of oligonucleotides, the 4 nucleic acids, A, T, C, and G, are added one by one to form a growing chain of nucleotides in the 3′ to 5′ direction. The nucleotides are built on a phosphoramidite building block. After its cleavage from the resin, the oligonucleotide still has a significant number of impurities. This is because, during synthesis, there are also shorter chains or failure sequences that arise. If the downstream application requires only the full-length sequence, then these shorter chains and failure sequences must be removed to create a purer oligo. Typically, to remove these shorter chains and failure sequences, the oligonucleotide crude material is subjected to one of reversed-phase high performance liquid chromatography (RP-HPLC), ion exchange chromatography (IEX), and hydrophobic interaction chromatography (HIC).

After the oligo is completed, it is typically concentrated by removal of the salts used in the chromatography process. Ultrafiltration (UF) and diafiltration (DF) are typically used for concentrating the product, removing salts, heavy metals and other small molecules, and exchanging the solvent. Conventionally, regenerated cellulose membranes have been used for the UFDF process. Regenerated cellulose membranes are hydrophilic and have a tight microstructure, as shown in FIG. 2, to provide for high retention of the oligonucleotide product. For the UFDF process, the regenerated cellulose membranes are typically packed in a plate and frame design in a cassette, as shown in FIG. 3. Screens are provided between the membrane layers to create flow turbulence to decrease fouling of the membranes through product accumulation on the membrane surface.

However, regenerated cellulose membranes can be very costly and have performance limitations. They primarily separate molecules based on the size of the molecule. Even though these membranes may have a relatively narrow or tight pore size distribution, there is still a small fraction of large pores that can allow the passage of the valuable oligomer molecules during the UF and DF steps. Another key feature of any membrane used in the UF/DF steps is the solution throughput rate, or permeation rate. Regenerated cellulose membranes have become a standard in the UF/DF concentration of oligomers, but the permeation rate with these commercial membranes still results in very long processing times. It is possible to achieve relatively higher throughput rates using regenerated cellulose UF membranes having larger pore sizes, but the trade-off for higher throughput is increased losses of the valuable oligomer product.

Thus, there is a need for a process for the concentration and diafiltration of oligonucleotides which is less costly and time-consuming than conventional methods, but which still achieves relatively low product loss.

The method of the present invention fulfills this need. The new method achieves improved yield and reduced material and labor costs, and is easily adaptable into existing facilities for large scale production, while also having improved overall recovery and high purity of the oligonucleotide. In particular, the present invention results in a 50%-90% reduction in costs (at least 50% reduction of raw material costs and at least 24% reduction of labor costs), at least 50% reduction in product loss, and higher throughput which results in about a 50% reduction in cycle time.

### BRIEF SUMMARY OF THE INVENTION

In one embodiment, the present invention relates to a method for concentration of oligonucleotides from a solution comprising negatively charged oligonucleotides. The method comprises the steps of circulating the solution through an ultrafiltration or nanofiltration unit comprising a membrane: filtering the solution through the membrane to remove salts from the solution and obtain a retentate solution and a permeate solution, wherein the oligonucleotides are retained in the retentate solution and the removed salts are contained in the permeate solution: diafiltering the retentate solution with a diafiltration buffer to produce a concentrated oligonucleotide solution: and collecting the concentrated oligonucleotide solution. The membrane has a nominal molecular weight cutoff in the range of from about 700 daltons to about 5000 daltons, a negatively charged surface with a zeta potential of −20 mV or lower, and a water flux in the range of 800 to 1500 ml/min/m².

Advantageous refinements of the invention, which can be implemented alone or in combination, are specified in the dependent claims. Features and details that are described in the context of the method shall also apply in relation to the apparatus and system used to carry out the method, and vice versa.

In summary, the following embodiments are proposed as particularly preferred in the scope of the present invention:

Embodiment 1: A method for concentration of oligonucleotides from a solution comprising negatively charged oligonucleotides, the method comprising the steps of: circulating the solution through an ultrafiltration or nanofiltration unit comprising a membrane having a nominal molecular weight cutoff in the range of from about 700 daltons to about 5000 daltons, a negatively charged surface with a zeta potential of −20 mV or lower, and a water flux in the range of 800 to 1500 ml/min/m²: filtering the solution through the membrane to remove salts from the solution and obtain a retentate solution and a permeate solution, wherein the oligonucleotides are retained in the retentate solution and the removed salts are contained in the permeate solution: diafiltering the retentate solution with a diafiltration buffer to produce a concentrated oligonucleotide solution: and collecting the concentrated oligonucleotide solution.

Embodiment 2: The method according to the preceding embodiment, wherein the negatively charged oligonucleotides comprise from about 5 to about 300 nucleotides.

Embodiment 3: The method according to the preceding embodiment, wherein the negatively charged oligonucleotides comprise from about 8 to about 50 nucleotides.

Embodiment 4: The method according to the preceding embodiment, wherein the negatively charged oligonucleotides comprise from about 15 to about 30 nucleotides.

Embodiment 5: The method according to the preceding embodiment, wherein the negatively charged oligonucleotides comprise from about 18 to about 25 nucleotides.

Embodiment 6: The method according to any of the preceding embodiments, wherein the negatively charged oligonucleotides have a zeta potential of from about −1 to −500 mV.

Embodiment 7: The method according to any of the preceding embodiments, wherein circulating of the solution through the ultrafiltration or nanofiltration unit and filtering of the solution is by tangential flow filtration.

Embodiment 8: The method according to any of the preceding embodiments, wherein prior to circulating of the solution through the ultrafiltration or nanofiltration unit, the ultrafiltration or nanofiltration unit is flushed with purified water at a cross flow rate of 2 to 10 L/min/m² and an average transmembrane pressure of 20 to 60 psi.

Embodiment 9: The method according to any of the preceding embodiments, wherein the solution is fed onto the membrane at a cross-flow rate of 2 to 10 L/min/m² and average transmembrane pressure of 20 to 60 psi.

Embodiment 10: The method according to the preceding embodiment, wherein the solution is fed onto the membrane at a cross-flow rate of approximately 4 to 6 L/min/m² and an average transmembrane pressure of approximately 35 psi.

Embodiment 11: The method according to any of the preceding embodiments. wherein an oligonucleotide concentration of the solution is 20 to 200 OD/mL and wherein an oligonucleotide concentration of the concentrated oligonucleotide solution is up to 4,000 to 8,000 OD/mL, the concentrations being measured in Optical Density (OD) per ml by UV analysis.

Embodiment 12: The method according to any of the preceding embodiments, wherein the solution is filtered until concentrated to a volume concentration of 2.5% to 25%.

Embodiment 13: The method according to any of the preceding embodiments, wherein the method is carried out until the conductivity of the permeate solution is from 10 to 200 micro-siemens per cm.

Embodiment 14: The method according to any of the preceding embodiments, wherein the membrane is selected from the group consisting of a flat plate device, a flat sheet cassette, a spiral wound cartridge, a hollow fiber device, a tubular device and a single sheet device.

Embodiment 15: The method according to the preceding embodiment, wherein the membrane is a spiral wound cartridge.

Embodiment 16: The method according to any of the preceding embodiments, wherein the membrane is a composite semipermeable membrane comprising a polyester web, a polysulfone substrate cast on the polyester web, and a sulfonated polyether sulfone surface layer on the polysulfone substrate.

Embodiment 17: The method according to the preceding embodiment, wherein the sulfonated polyether sulfone surface layer has a thickness of 0.3 microns.

Embodiment 18: The method according to any of the preceding embodiments, wherein the membrane has a nominal molecular weight cutoff (MWCO) of around 720 daltons to 5 KD.

Embodiment 19: The method according to the preceding embodiment, wherein the nominal MWCO of the membrane is around 1 KD to 3 KD.

Embodiment 20: The method according to any of the preceding embodiments, wherein the membrane has a negative surface charge.

Embodiment 21: The membrane according to the preceding embodiment, wherein the sulfonated polyether sulfone surface layer of the membrane has a zeta potential of approximately −10 mV or greater negative charge.

Embodiment 22: The membrane according to the preceding embodiment, wherein the sulfonated polyether sulfone surface layer of the membrane has a zeta potential of approximately −20 mV or greater negative charge.

Embodiment 23: The membrane according to the preceding embodiment, wherein the sulfonated polyether sulfone surface layer of the membrane has a zeta potential of approximately −30 mV or greater negative charge.

### BRIEF DESCRIPTION OF THE DRAWINGS

The patent or application file contains at least one drawing executed in color. Copies of this patent or patent application publication with color drawings will be provided by the Office upon request and payment of the necessary fee.

The following detailed description of preferred embodiments of the present invention will be better understood when read in conjunction with the appended drawings. For the purposes of illustrating the invention, there are shown in the drawings embodiments which are presently preferred. It is understood, however, that the invention is not limited to the precise arrangements and instrumentalities shown. In the drawings:

FIGS. 1A and 1B are schematic diagrams of the manufacturing process for RNA and DNA oligonucleotides, respectively:

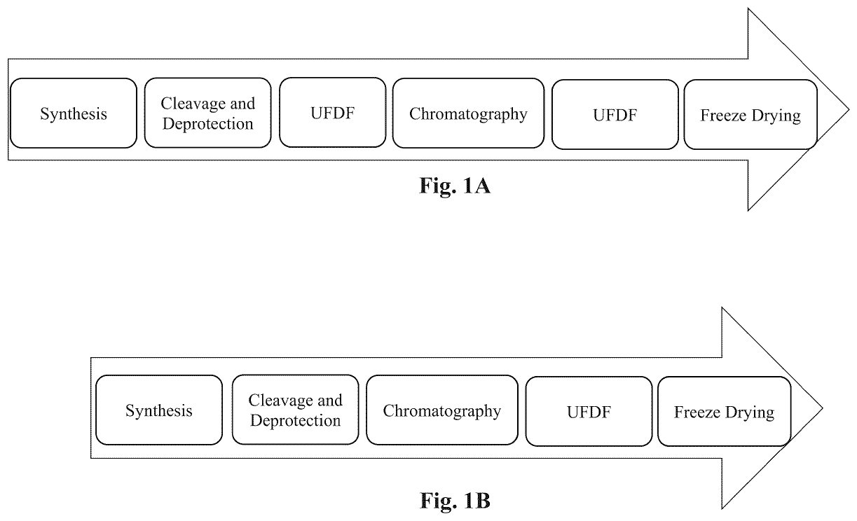

FIG. 2 depicts a prior art regenerated cellulose membrane:

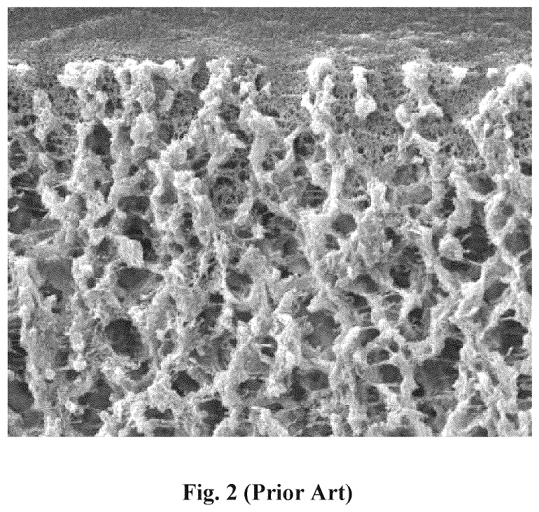

FIG. 3 depicts prior art regenerated cellulose membranes packed in a plate and frame design in a cassette:

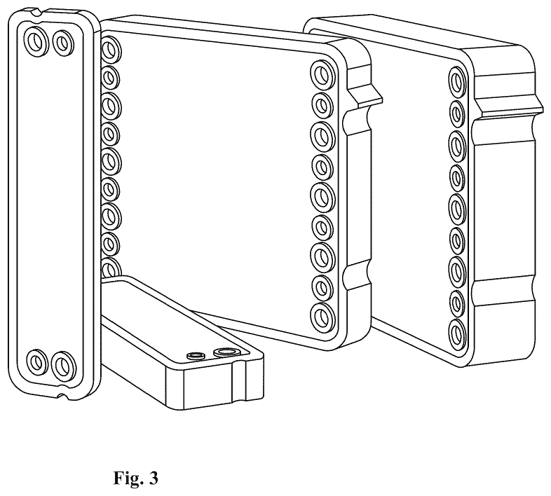

FIGS. 4A and 4B depict a negatively charged oligonucleotide molecule strand according to embodiments of the present invention, with a negative field charge being pictorially depicted in FIG. 4A:

FIG. 5 graphically depicts the zeta potential measurement of an exemplary oligonucleotide molecule according to embodiments of the present invention:

FIG. 6A is a schematic diagram of a pre-filtration flushing process and FIG. 6B is a schematic diagram of tangential flow ultrafiltration of an oligonucleotide solution, according to embodiments of the present invention:

FIG. 7 is a schematic depiction of a spiral wound module used in embodiments of the present invention:

FIG. 8 shows a spiral wound module, used in embodiments of the present invention, in a partially unwound configuration:

FIG. 9 depicts a partial cross-sectional view of a sulfonated polyether sulfone composite membrane used in embodiments of the present invention:

FIG. 10 depicts the zeta potential of various membranes according to embodiments of the present invention:

FIG. 11 compares pore volume v. pore size of a sulfonated polyether sulfone composite membrane used in embodiments of the present invention and a prior art regenerated cellulose membrane: and

FIG. 12 provides a statistical data analysis of different sample lots of the 24-mer oligonucleotide versus the total oligonucleotide concentration loaded on a regenerated cellulose membrane having a surface area of 2.5 m² w.

### DETAILED DESCRIPTION OF THE INVENTION

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The invention generally relates to a method for preparing concentrated oligonucleotides from a solution comprising negatively charged oligonucleotides. The method comprises ultrafiltration and diafiltration steps using a membrane having an enhanced negatively charged surface, a larger pore size than the oligonucleotides and a high pore volume in the ultrafiltration range.

“Ultrafiltration” refers to a technique to separate particles or molecules by filtration through membranes having pore sizes ranging from about 0.005 μm to about 0.1 μm.

“Diafiltration” is a mode of operating an ultrafiltration system in which the retentate is continuously concentrated, recycled and diluted with fresh wash solution to replace that which is removed as permeate. Diafiltration will generally provide a cleaner separation of macromolecules retained in the retentate sample while the smaller molecules pass through into the filtrate. Diafiltration may be carried out in a batch mode or a continuous mode. Continuous diafiltration refers to the continuous addition of fresh wash buffer as filtration takes place. Batch mode diafiltration refers to the repeated steps of concentrating the sample by ultrafiltration and then diluting with buffer.

“Permeate” or “filtrate” refers to that portion of a sample that passes through the membrane, such as molecules which are smaller than the membrane pores and are therefore allowed to pass through the membrane pores.

“Retentate” refers to that portion of a solution that does not pass through the membrane, such as molecules that are larger than the membrane pores.

“Tangential flow” or “cross-flow” filtration refers to a filtration process in which the sample solution circulates across the top of the membrane, while applied pressure causes solute and small molecules to pass through the membrane.

A “feed solution” refers to any solution that contains a compound to be concentrated.

The present invention utilizes ultrafiltration, and more preferably tangential flow ultrafiltration, and diafiltration for concentration of oligonucleotides. However, it will be understood by those skill in the art that the method of the present invention may instead utilize dead end filtration or other known filtration techniques (e.g., filtration systems having vibrating membranes, rotating discs, and the like).

The oligonucleotides of the present invention include single-stranded and double-stranded oligonucleotides. The oligonucleotides of the present invention preferably comprise from about 5 to about 300 nucleotides, more preferably from about 8 to about 50 nucleotides, more preferably from about 15 to about 30 nucleotides, with 18 to 25 nucleotides being particularly preferred. In one embodiment, the oligonucleotides comprise from 15 to 18 nucleotides. In another embodiment, the oligonucleotides comprise 18 to 24 nucleotides. In one embodiment, the oligonucleotides comprise 18 nucleotides. In one embodiment, the oligonucleotides comprise 20 nucleotides. In another embodiment, the oligonucleotides comprise 23 nucleotides. In another embodiment, the oligonucleotides comprise 24 nucleotides.

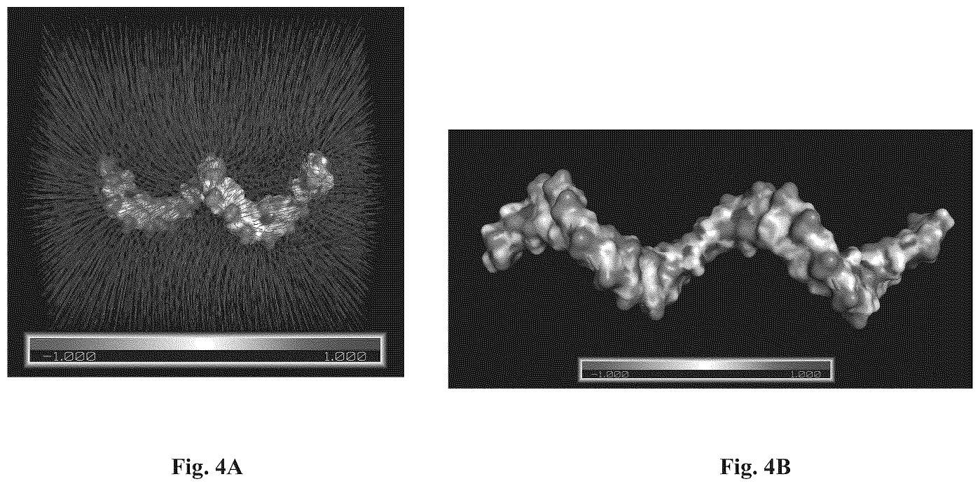

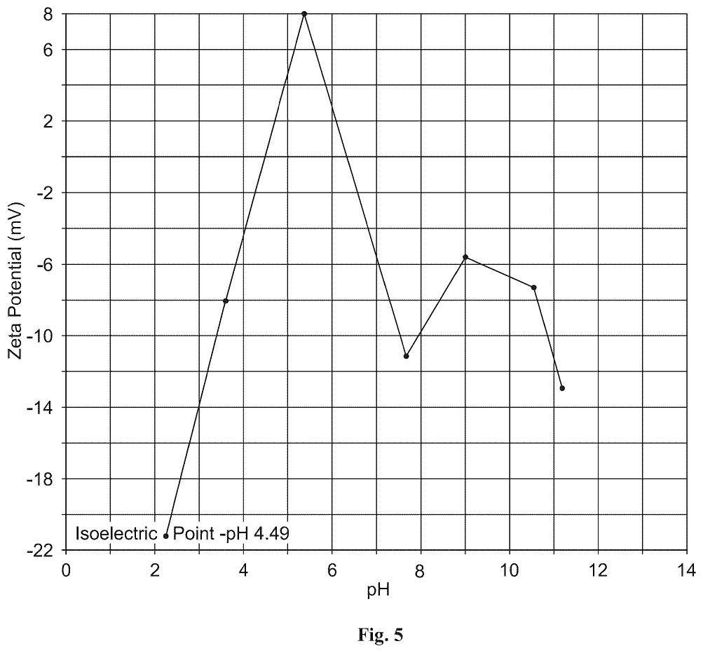

The oligonucleotide molecules of the present invention are preferably negatively charged as depicted in FIGS. 4A and 4B. Referring to FIG. 4A, there is depicted a strong negative charge field around the oligonucleotide, with the negative charges being shown in red. FIG. 5 graphically depicts the zeta potential measurement of an exemplary oligonucleotide molecule according to the present invention. Preferably, the oligonucleotides of the present invention have a zeta potential of from about −1 to −500 mV. It will be understood by those skilled in the art that the degree of negative charge of the molecules will depend on various factors, such as the number of nucleotides, the solution composition, if any backbone modifications have been made, and the like. In one embodiment, a 23-mer oligonucleotide has a zeta potential of −56 mV. In another embodiment, a 24-mer oligonucleotide has a zeta potential of −48 mV.

In one embodiment, the oligonucleotides have a linear chain. However, it will be understood by those skill in the art that the oligonucleotides may have a different structure, such as comb-like, circular and the like, and other add-on molecules.

The method of the present invention comprises concentrating these negatively charged oligonucleotides by tangential flow ultrafiltration combined with diafiltration, such that the oligonucleotide feed solution is concentrated and/or exchanged into a buffer solution in order to remove solvents, salts and other small molecules.

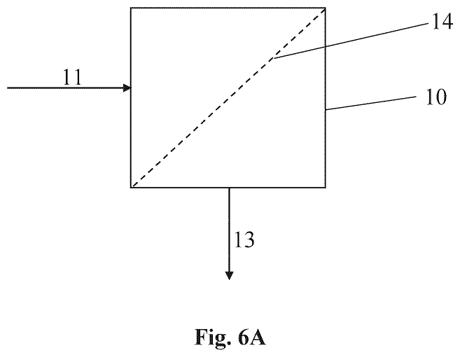

FIGS. 6A-6B show schematic diagrams of pre-filtration and tangential flow ultrafiltration of an oligonucleotide solution, respectively, according to the present invention, although it will be understood by those skilled in the art that the present invention may be performed using other filtration techniques and systems. As is well known in the art, tangential flow filtration is a filtration technique in which the feed solution passes tangentially along the surface of the membrane, such that the fluid flow along the membrane surface sweeps away residue buildup on the membrane surface and reduces fouling of the membrane, while the retentate solution can easily be recirculated, thereby allowing thorough processing of large volumes of solution. More particularly, during tangential flow filtration, pressure is applied across the membrane, which will allow smaller molecules, and more particularly the molecules having a smaller molecular weight than the membrane pores, to pass through the membrane while the molecules having a larger molecular weight than the membrane pores are retained above the membrane as retentate and recirculated. A circulation pump (not shown) may be included in the feed line to control the solution flow. The circulation pump may be a peristaltic pump, a diaphragm pump or the like.

Referring to FIGS. 6A and 6B, the process according to the present invention utilizes a filtration unit 10 fitted with a membrane 14. In one embodiment, the filtration unit 10 preferably comprises a stirred cell apparatus configured for tangential flow operation. The filtration unit 10 may be an ultrafiltration or nanofiltration unit.

Referring to FIG. 6A, before introduction of the oligonucleotide feed solution into the filtration unit 10, the filtration unit 10 is preferably flushed with purified water (represented by water line 11) at a target flow rate for a predetermined duration (e.g., approximately one hour) to wet and pre-condition the membrane 14 and remove any storage solution. The pre-filtration flush is preferably carried out at a cross flow rate of 2 to 10 L/min/m², more preferably approximately 4 to 6 L/min/m², and most preferably approximately 5 L/min/m², and an average transmembrane pressure of 20 to 60 psi, preferably 30 to 35 psi, most preferably approximately 35 psi. The flushed out water is shown as stream 13 in FIG. 6A.

Referring to FIG. 6B, there are shown two steps of tangential flow ultrafiltration of an oligonucleotide solution, according to an embodiment of the present invention. In Step 1. the oligonucleotide feed solution enters the filtration unit 10 through a feed channel or feed line 12. As the oligonucleotide feed solution is introduced into the filtration unit 10, the feed solution flows along the surface of the membrane 14 and pressure is applied across the membrane 14, such that the smaller molecules pass through the membrane 14. The feed solution is fed onto the membrane 14 at a cross-flow rate of 2 to 10 L/min/m², more preferably approximately 4 to 6 L/min/m², and most preferably approximately 5 L/min/m². The oligonucleotide concentration of the feed solution is preferably 20 to 200 OD/mL, more preferably 80 to 130 OD/mL (the concentration being measured in Optical Density (OD) per ml by UV analysis). An average transmembrane pressure of 20 to 60 psi, preferably 30 to 35 psi, most preferably approximately 35 psi is maintained throughout the UFDF process. The average transmembrane pressure may be controlled, for example, by adjustment of a valve or a pump (not shown) in the retentate line 18, or by maintaining a constant pressure and/or volume across the membrane 14. As such, the membrane 14 of the filtration unit 10 separates the oligonucleotide feed solution into a permeate solution (represented by permeate stream 16) containing the oligonucleotide and a retentate solution (represented by retentate stream 18) containing unwanted salts.

The permeate solution exits the filtration unit through a permeate channel or permeate line 16. The concentrated retentate solution passes into a retentate channel or retentate line 18, which is circulated back into the feed line 12 for diafiltration or which proceeds to the next stage/step of the manufacturing process if it has been concentrated to the predetermined concentration. Preferably, the oligonucleotide feed solution is initially filtered without buffer addition until concentrated up to an initial concentration of 4,000 to 8,000 OD/mL, preferably up to 450 to 750 OD/mL and a volume concentration of 2.5% to 25%, preferably approximately 2.5%. It will be understood that the desired volume and initial concentration may vary depending upon the product to be produced and the desired properties thereof.

In Step 2, once concentrated to the desired volume and initial concentration, diafiltration buffer is added via a buffer line 20, and filtration continues to wash the retentate solution of contaminating small molecules and to remove unwanted solvents and salts. Diafiltration may be carried out in a continuous mode or batch mode. Preferably, diafiltration is continuous and is performed until about 7 to about 500 volume equivalents have been exchanged, preferably at least 7 volume equivalents. Generally, more or less diafiltration will be performed with increased or decreased contaminants bound to the nucleic acids, depending upon the purity required for the end application. At the end of diafiltration, the product is preferably concentrated up to 4,000 to 8,000 OD/mL, more preferably up to 450 to 750 OD/mL, and a volume concentration of 2.5% to 25%, preferably approximately 2.5%.

The UFDF process is carried out until the conductivity of the permeate solution, which is a measure of the salt content of the permeate solution, is from 10 to 200 micro-siemens per cm, and most preferably approximately 50 micro-siemens per cm, as measured by a conductivity meter.

It will be understood by those skilled in the art that any known controls, such as pressure transducers, flow meters, in-line conductivity/pH meters, sensors, valves and the like may be employed in the feed line, permeate line, retentate line, and/or buffer line for controlling flow through the respective line.

The buffer may be purified water or sodium phosphate at a pH of 6.8-7.2. It will be understood by those skilled in the art that any buffer solution having a similar pH (i.e., 6.8-7.2) may be utilized.

The membrane utilized in the method of the present invention may have a variety of known configurations, such as a flat plate device, spiral wound cartridge, hollow fiber, tubular or single sheet device. Preferably, the membrane is a spiral wound cartridge which enables a higher contact area with the solution to be filtered and thereby reduces cycle time. However, it will be understood by those skilled in the art that other configurations, namely a flat sheet or cassette, may be utilized.

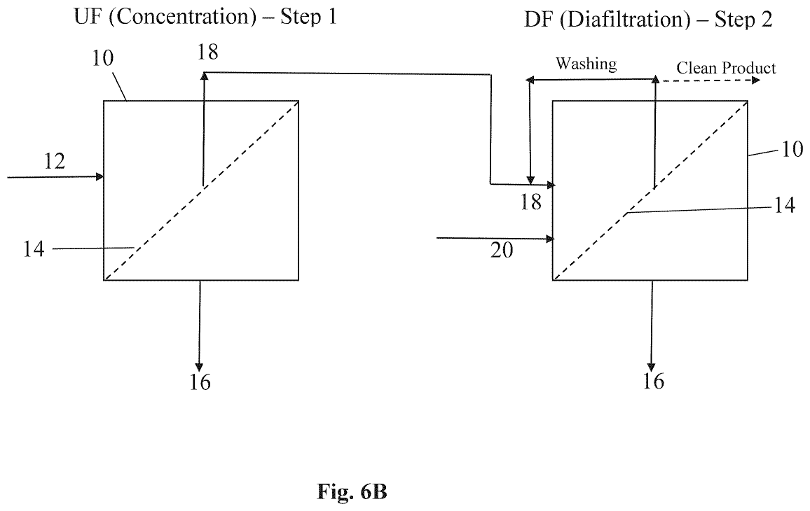

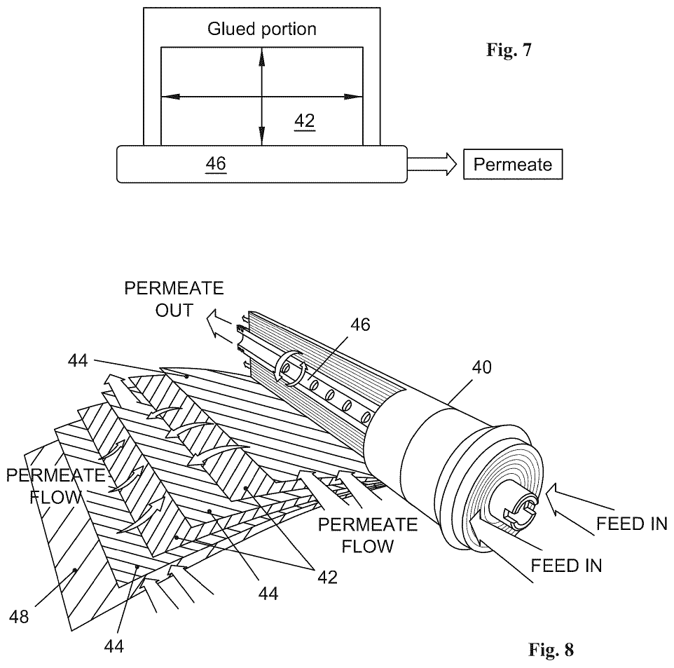

A spiral wound module 40, as depicted in FIGS. 7-8, comprises membranes 42, feed (or permeate) spacers 44, and a permeate (or core) tube 46. To form the spiral wound module 40, a membrane 42 is first laid out and folded in half with the membrane 42 facing inward. A feed spacer 44 is then put in between the folded membrane 42, essentially forming a membrane sandwich. The purpose of the feed spacer 44 is to provide space for the feed solution to flow between the membrane 42 surfaces, and to allow for uniform flow between the membrane 42 leaves. Next, a permeate spacer 44 is attached to a permeate tube 46, and the membrane sandwich prepared earlier is attached to the permeate spacer 44, for example, using an adhesive. Next, a permeate layer is laid down and sealed with adhesive, and process is repeated until all of the required permeate spacers 44 have been attached to the membranes 42. The finished membrane layers then are wrapped around the permeate tube 46 creating the spiral shape. The assembly includes an outer wrap 48.

During filtration, the feed solution travels through the flow channels tangentially across the length of the element 40. Filtrate smaller than the membrane molecular weight cutoff (MWCO) will pass across the membrane 42 surface into the permeate spacer 44, where it is carried down the permeate spacer 44 towards the permeate tube 46 and extracted from the system as permeate. The remainder of the feed solution becomes concentrated retentate at the end of the membrane element 40.

The dimensions and active surface area of the membrane used will depend on the volume of oligonucleotide to be processed. More particularly, the surface area of the membrane element will be selected based on the flow rates and drug volumes to be processed. For example, a feed rate of 8 L/m may be treated by a membrane element having 2.5 m² of surface area, to produce 1 L/m of permeate and 7 L/m of retentate. A 2.5 m² spiral element which could process 8 L/m of feed flow, in accordance with an embodiment of the present invention, would ideally be spirally wound, with the spiral wound element with a diameter of 2.5 inches and a length of 40 inches. Larger flow rates, such as those of commercial operations, would require a larger membrane area. For example, a feed flow of 60 L/m may be treated by utilizing a plurality (e.g., up to 3) of membrane elements, each having a 7 m² surface area, which are wound in a spiral configuration. For a feed flow rate of 20 L/m, the process may be carried out by a spiral wound element 4 inches in diameter and 40 inches long, which would produce 2.4 L/m per element. For smaller feed flows, the process may be carried out by a spiral wound element 1.8 inches in diameter and 12 inches long elements (with a membrane area of 0.4 m²). Larger flows may be treated with a spiral wound element 8 inches in diameter and 40 inches long (with 37 m² of membrane area). It will be understood by those skilled in the art that the above are just examples of the different membrane areas and configurations that may be used, and that any appropriate configuration and membrane area may be selected based on the feed volume and flow rate.

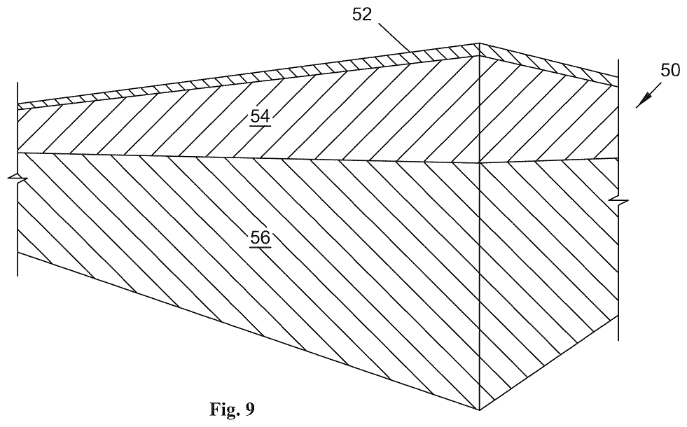

The membrane to be used in the present invention is preferably chemical and oxidant resistant. The membrane to be used in the present invention also preferably meets all relevant guidelines and quality assurance requirements of the Current Good Manufacturing Practice (cGMP) regulations. The membrane to be used in the present invention is preferably a composite semipermeable membrane, and more preferably a sulfonated polyether sulfone composite semipermeable membrane. More particularly, the surface material of the membrane is preferably sulfonated polyether sulfone. In one embodiment, as depicted in FIG. 9, the present invention utilizes a membrane 50 comprises a sulfonated polyether sulfone coating 52 on a polysulfone substrate 54 cast on a polyester web 56, referred to herein as an SPES composite membrane 50. Preferably, the top sulfonated polyether sulfone 52 has the following chemical structure:

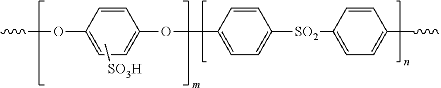

In one embodiment, the membrane 50 is preferably a sulfonated polyether sulfone composite semipermeable membrane as disclosed in EP 0165077B2, the entire contents of which are incorporated herein by reference.

The membrane utilized in the method of the present invention is suitable for separating the desired oligonucleotide from the undesired components or contaminants, such as salts, heavy metals, failure sequences that form during synthesis, and other small molecules, of the feed solution from which the oligonucleotide is to be concentrated. The separation is performed by the top sulfonated polyether sulfone layer. In a preferred embodiment, as shown in FIG. 9, the sulfonated polyether sulfone layer has a thickness of 0.3 microns. As such, the pores of the sulfonated polyether sulfone layer are less than 0.3 microns, with the pores of 0.1 micron or smaller performing ultrafiltration of the feed and retentate solution for separation of the negatively charged oligonucleotides from the undesired components or contaminants. In the membrane 50 of FIG. 9, the polysulfone layer 54 preferably has a thickness of 50 microns, and the polyester base 56 preferably has a thickness of 150 microns.

Oligonucleotides according to the present invention have a molecular weight of 1 KD to 500 KD. For concentration and diafiltration of oligonucleotides according to the present invention, the membrane preferably has a nominal MWCO of around 700 daltons to 5 KD, more preferably a nominal MWCO of around 720 daltons to 5 KD, more preferably a nominal MWCO of around 720 daltons to 3 KD, and even more preferably a nominal MWCO of around 1 KD to 3 KD. It will be understood by those skilled in the art that MWCO is measured as being 90% of a certain MW molecule being rejected, but the charge, size and shape of those molecules can affect the rejection.

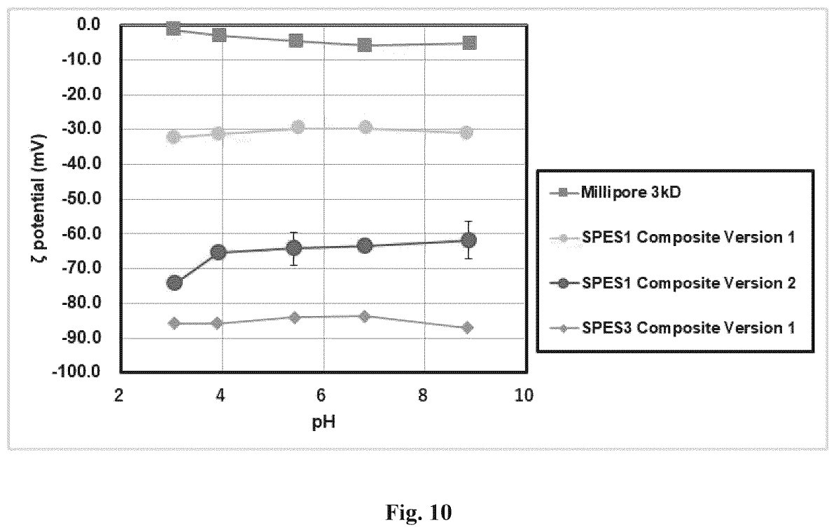

The membrane also preferably has a negative surface charge. The zeta potential of the surface of various sulfonated polyether sulfone (SPES) composite semipermeable membranes, in accordance with one embodiment of the present invention, as compared with that of a conventional regenerated cellulose membrane, is shown in FIG. 10. The sulfonated polyether sulfone composite semipermeable membranes are labelled as “SP1S* Composite” in FIG. 10. Referring to FIG. 10, the regenerated cellulose membrane (labelled as “Millipore 3 kD”), and the SPES1 Composite Version 1 membrane and the SPES1 Composite Version 2 membrane have MWCO of 3 KD.

As shown in FIG. 10, the membrane, and more preferably the surface of the SPES composite membrane, for use in the method of the present invention has a zeta potential of approximately −10 mV or greater negative charge, preferably approximately −20 mV or greater negative charge, more preferably approximately −30 mV or greater negative charge. In some cases, the negative charge can be as great as −90 mV.

Due to the strong negative charge of the sulfonated polyether sulfone material and the relative small passageways (i.e., pores) through the membrane, there is strong and close interaction of the negatively charged oligonucleotide molecules with the SPES polymer. As a result of this strong interaction between the sulfonate groups and the negative charges on the oligomers, there is a strong repulsion force preventing the oligomer from entering the pores of the SPES layer. As a result of this interaction, and more particularly this repulsion effect. during the concentration and diafiltration steps, the oligonucleotide molecules are retained in the retentate, with minimal loss through the membrane to the permeate side of the membrane. The retention of the oligonucleotide molecules is increased and the loss of these molecules through the permeate is reduced as with the UFDF process carried out with a regenerated cellulose membrane.

The sulfonated polyether sulfone material gives the membranes unique proton (hydrogen ion) transport properties. More particularly, the sulfonated molecules are preferably arranged in a way which creates channels with sulfonate groups and which gives the membrane unique transport properties. As a result, there is a great degree of interaction between the negative charges of sulfonate groups and the oligonucleotide molecules. In one embodiment, the membrane according to the present invention also has an ion exchange capacity of approximately 1.16 meq/g to 1.36 meq/g.

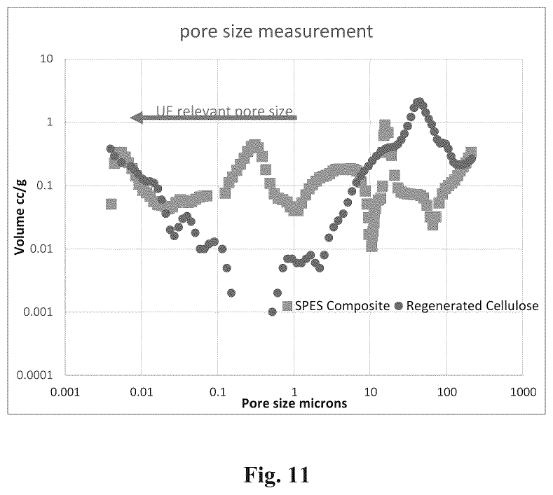

The membrane used in the method of the present invention preferably has a relatively broad pore size distribution. More particularly, the sulfonated polyether sulfone top layer of the composite membrane preferably has a relatively high volume of pores of less than 0.3 microns, and more particularly of pores of 0.1 micron or less (i.e., pore sizes in the ultrafiltration range). For example, as shown in FIG. 11, which shows the distribution of pores throughout the cross-section of the composite membrane for a SPES composite nominally 3 KD membrane, a SPES composite nominally 3 KD membrane according to the present invention, preferably has larger sized pores and a higher volume of pores of 0.3 microns or less, and more preferably 0.1 microns or less, than a regenerated cellulose 3 KD membrane, and more particularly in the UF range, the volume of pores is up to 3 degrees of magnitude larger than a regenerated cellulose 3 KD membrane.

Thus, referring to FIG. 11, it can be seen that the SPES composite nominally 3 KD membrane is more porous than the regenerated cellulose membrane having a nominal MWCO of 3 KD, and more particularly has larger pores overall and is more porous in the ultrafiltration range than the regenerated cellulose membrane having a nominal MWCO of 3 KD. As a result, the SPES composite membrane exhibit higher flux performance as compared to a regenerated cellulose membrane. Preferably, the water flux of the membrane, and more particularly the SPES composite membrane, is 800 to 1500 ml/min/m².

Further, due to the negative surface charge of the SPES composite membranes, even though there is increased flux across the membrane, the inventors of the present invention have found that the SPES composite membranes are still very selective to prevent loss of the oligomer. More particularly, the negative surface charge of the SPES composite membrane has a strong interaction with the negatively charged oligonucleotides, which causes the oligonucleotide molecules to be repelled by the membrane surface. The combination of the larger pore sizes, high pore volume in the ultrafiltration range, and the negatively charged membrane surface therefore enables high flux across the membrane with minimal loss of the oligonucleotide molecules through the permeate.

Also, as a result of the relatively higher flux of the permeate, overall cycle times are reduced by the method of the present invention as compared with conventional methods. It will be understood by those skilled in the art that, ultimately, cycle times will vary depending upon the amount of contaminants bound to the nucleic acids and the purity required for the end application of the oligonucleotides.

The present invention is illustrated by the following non-limited examples.

### EXAMPLES

Five different types of membranes were tested as follows:
Membranes SPES1a, SPES1b and SPES1b: spiral wound sulfonated polyether sulfone composite membrane (e.g., Hydranautics HydraCoRe);
Membrane PPZ2: spiral wound piparazine thin-film composite membrane (e.g., Microdyn Nadir's Trisep UA60):
Membrane PES3: spiral wound polyether sulfone membrane (e.g., Synder VT):
Membrane RCS4: spiral wound regenerated cellulose membrane (e.g., Millipore flat sheet membrane rolled as a spiral element); and
Membrane RCF5: regenerated cellulose membrane in flat sheet configuration (e.g., Millipore flat sheet membrane).

Additional characteristics of each membrane are provided in Table 1.

TABLE 1
Details of Membranes Evaluated

| | | | | Surface | |
|---|---|---|---|---|---|
| | | | | area | Zeta |
| Membrane | Materials | Configuration | MWCO | (m²) | Potential |
| SPES1a | Sulfonated | Spiral Wound | 3KD | 0.352 | −60 mV to |
| SPES1b | Polyether | | | 0.1 | −70 mV |
| SPES1c | Sulfone | | | 0.04 | |
| | composite | | | | |
| PPZ2 | Piparazine | Spiral Wound | 1KD | 0.23 | Neutral |
| | composite | | | | |
| PES3 | Polyether | Spiral Wound | 3KD | 0.33 | Neutral |
| | Sulfone | | | | |
| RCS4 | Regenerated | Spiral Wound | 3KD | 0.352 | 0 mV to - |
| | Cellulose | | | | 10 mV |
| RCF5 | Regenerated | Flat Sheet | 3KD | 2.5 | 0 mV to - |
| | Cellulose | Cassette | | | 10 mV |

Membrane RCF5 is the type of membrane conventionally used in the UFDF process for oligomers, and was included as a basis for comparison.

Three different length oligonucleotides were tested for evaluation of each membrane as follows: a 24-mer oligonucleotide product was tested on each membrane listed in Table 1: a 23-mer oligonucleotide product was tested on the SPES1a membrane; and a 18-mer oligonucleotide product was tested on the SPES1c membrane. The tested oligonucleotides were all different versions of a commercially available oligonucleotide product, CPG 7909.

Each membrane listed in Table 1 was tested in a test cell manufactured and sold by either Sterlitech or Smartflow (e.g. Smartflow PuroSep Pegasus) for concentration and diafiltration of the 24-mer oligonucleotide, 23-mer oligonucleotide and/or 18-mer oligonucleotide, as indicated above. A spiral membrane module having a length of 18 inches and a diameter of 1.2 inches was used to test each spiral wound membrane.

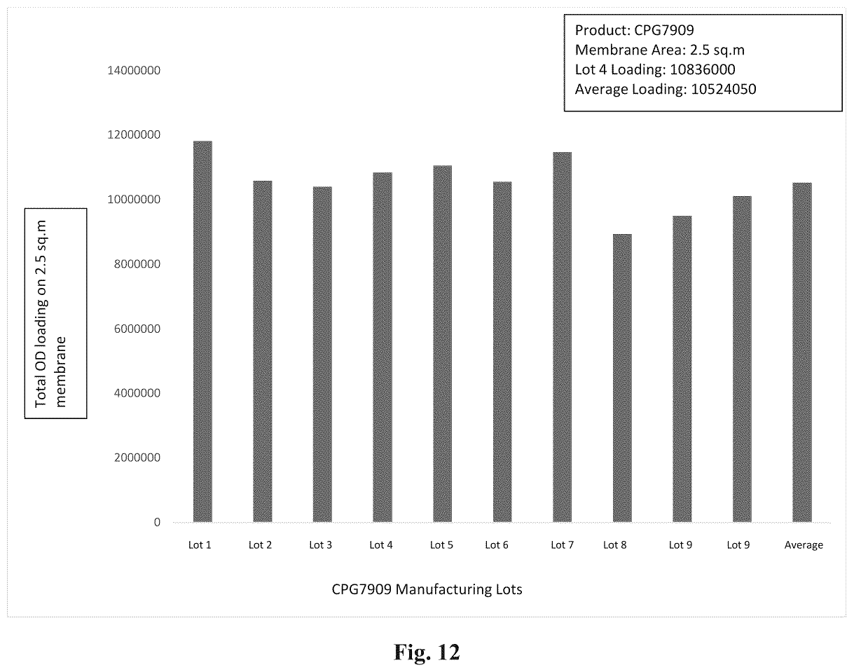

The experiments were designed to closely replicate the UFDF operation in a commercial manufacturing process. The process parameters of the UFDF were selected to mimic commercial manufacturing process conditions by scaling down the commercial manufacturing process parameters. A statistical data analysis of different sample lots of the 24-mer oligonucleotide versus the total oligonucleotide concentration loaded on a RCF5 membrane having a surface area of 2.5 m² was prepared as shown in FIG. 12. The average loading on the membrane for all of the samples was closest to the loading value of the sample corresponding to Lot No. 100006. Accordingly, the average OD loading selected for the experiments was that of the sample of Lot No. 100006, specifically 10524050. The remaining UFDF process parameters for a 2.5 m² membrane are shown below in Table 2.

TABLE 2
UFDF manufacturing process parameters

| | Process Parameter | |
|---|---|---|
| Process Parameter | absolute value | |
| Membrane surface area | 2.5 | m² |
| Membrane MWCO | 3 | KD |
| Total Feed OD | 16335000 | |
| Concentration Factor | 4 | |
| Transmembrane Pressure | 35 | PSI |
| Cross-Flow rate | 5 | L/min/m² |

The process parameters listed above in Table 2 were then scaled down appropriately to determine the appropriate oligomer loading and the cross-flow rate required for each membrane surface area listed in Table 1, for laboratory testing purposes. For example, as shown in FIG. 12, the average OD loading across a 2.5 m² membrane was found to be 10524050. Therefore, for a 0.352 m² membrane for purposes of laboratory testing, the scaled-down oligo loading is 1481786. Similarly, the cross-flow rate for a 2.5 m² membrane utilized in manufacturing is 5 L/min/m². The scaled-down cross-flow rate for a 0.352 m² membrane is therefore 1.76 L/min.

The scaled-down process parameters for each of the experiments carried out on membranes SPES1a, SPES1b, and SPES1c, which are preferred for use in the method of the present invention, are shown in Table 3.

TABLE 3
Scaled-Down Process Parameters for Membranes SPES1a, SPES1b, and SPES1c

| Process Parameter | SPES1a | SPES1b | SPES1c | PPZ2 | PES4 | RCS4 | RCF5 |
|---|---|---|---|---|---|---|---|
| Surface area (m²) | 0.352 | 0.1 | 0.04 | 0.23 | 0.33 | 0.352 | 2.5 |
| MWCO | 3KD | 3KD | 3KD | 1KD | 3KD | 3KD | 3KD |
| Total Feed (% OD) | 1481786 | 420961 | 168384 | | 1481786 | 1481786 | 16335000 |
| Concentration Factor | 4 | 4 | 4 | 4 | 4 | 4 | 4 |
| Transmembrane | 35 | 35 | 35 | 35 | 35 | 35 | 35 |
| Pressure (PSI) | | | | | | | |
| Cross-Flow rate | 1.76 | 0.5 | 0.2 | 1.15 | 1.76 | 1.76 | 5 |
| (L/min) | | | | | | | |
| Surface Charge | Negative | Negative | Negative | Neutral | Neutral | Slightly | Slightly |
| | | | | | | negative | negative |
| Zeta Potential (mV) | −60 to −70 | −60 to −70 | −60 to −70 | Neutral | Neutral | 0 to −10 | 0 to −10 |

To carry out the tests, each of the membranes SPES1a, SPES1b, SPES1c, PPZ2, PES3, RCS4 and RCF5 was connected to the test cell system and the system was flushed with purified water at a target flow rate of 5 L/min/m² for approximately one hour to wet the membrane and remove any storage solution. A pre-use water flux was measured for each experiment.

Then, for each experiment, either a 24-mer oligonucleotide, 23-mer oligonucleotide or 18-mer oligonucleotide feed solution was introduced across the membrane for UFDF thereof. The feed volume for each experiment was always greater than or equal to 4 L, so that the initial concentration volume, based on a concentration factor of 4, was always above 1 L (i.e., due to the pump's limitations of the test cell systems in handling low volumes). The UFDF process for each membrane was carried out in accordance with the process parameters set forth in Table 3.

After a concentration factor of 4 was achieved, the diafiltration operation was carried out with a minimum of 7 diafiltration exchanges, with each exchange volume being equivalent to the volume of concentrated product solution. Purified water was used as the diafiltration buffer. For each experiment, the UFDF process was stopped when the permeate conductivity was measured to be 50 micro-siemens per cm.

For each experiment, samples of the feed solution, concentration retentate, concentration permeate, diafiltration permeate and diafiltration retentate were collected. The results of each experiment were analyzed for flux normalized per square meter of the membrane area (ml/min/m²) and the total product loss through the permeate (calculated based on concentration measured in Optical Density (OD) per ml by UV analysis). The flux and product loss results for each experiment are shown in Tables 4-6.

TABLE 4
Results of 24-mer Oligonucleotide Concentration

| | Average Flux | | | Product Loss Through | |
|---|---|---|---|---|---|
| | (ml/min/m²) | | | Permeate (% OD) | |
| | | Concen- | | Concen- | |
| Membrane | Water | tration | Diafiltration | tration | Diafiltration |
| SPES1a | 927.72 | 418.98 | 555.23 | 1.30 | 0.74 |
| SPES1b | 833.33 | 697.72 | 673.73 | 1.36 | 1.84 |
| SPES1c | 1338.7 | 950.96 | 816.54 | N/A | N/A |
| PPZ2 | 621.11 | 349.14 | 315.60 | 0.52 | 0.17 |
| PES3 | 757.58 | 473.75 | 381.62 | 2.80 | 1.90 |
| RCS4 | NA252.97 | 169.66 | 158.78 | 2.90 | 4.40 |
| RCF5 | 272 | 237.23 | 257.58 | N/A | N/A |

* The water flux data is pre-use water flux.

TABLE 5
Results of 23-mer Oligonucleotide Concentration

| | Average Flux | | | Product Loss Through | |
|---|---|---|---|---|---|
| | (ml/min/m²) | | | Permeate (% OD) | |
| | | Concen- | | Concen- | |
| Membrane | Water | tration | Diafiltration | tration | Diafiltration |
| SPES1a | 946.97 | 535.63 | 388.19 | 0.11% | 0.19% |

* The water flux data is pre-use water flux.

TABLE 6
Results of 18-mer Oligonucleotide Concentration

| | Average Flux | | | Product Loss Through | |
|---|---|---|---|---|---|
| | (ml/min/m²) | | | Permeate (% OD) | |
| | | Concen- | | Concen- | |
| Membrane | Water | tration | Diafiltration | tration | Diafiltration |
| SPES1c | 833 | 822.22 | 744.22 | 0.707% | 1.19% |

* The water flux data is pre-use water flux.

Based on the collective data, the sulfonated polyether sulfone membranes SPES1a, SPES1b, and SPES1c, which have a negative surface charge, achieved effective ultrafiltration and diafiltration of the different sized negatively charged oligonucleotide products, and showed an improved performance when compared to the regenerated cellulose cassettes conventionally utilized.

With the negatively charged sulfonated polyether sulfone membranes SPES1a, SPES1b, and SPES1c, the water flux and permeate flux (in both the UF and DF steps) was found to be 2.5 to 3 times higher flow as compared to the other membranes. However, while one may have expected a higher flux to result in lower rejection or retention of the oligonucleotide molecules, it was surprisingly found that oligonucleotide loss through the permeate was actually reduced using the negatively charged sulfonated polyether sulfone membranes, as compared to the % permeate loss of the other membranes. More particularly, with the negatively charged sulfonated polyether sulfone membranes SPES1a, SPES1b, and SPES1c, the % permeate loss was found to be 2 to 3 times lower than that associated with the regenerated cellulose membranes. This is because the interaction between the negative surface charge of the SPES membranes and the negatively charged oligonucleotide molecules creates a repulsion effect, such that during both the concentration and diafiltration steps, the oligonucleotide molecules are retained in the retentate with minimal loss through the permeate, even with a relatively high permeate flux, large pore sizes and high porosity in the ultrafiltration range.

The method according to the present invention therefore achieves improved product yield at reduced cycle times, thereby reducing overall costs, while also having superior overall recovery and providing high purity of the oligonucleotide.

It will be appreciated by those skilled in the art that changes could be made to the embodiments described above without departing from the broad inventive concepts thereof. Also, based on this disclosure, a person of ordinary skill in the art would further recognize that the various ranges described above could be varied without departing from the spirit and scope of the invention. It is understood, therefore, that this invention is not limited to the particular embodiments disclosed, but it is intended to cover modifications within the spirit and scope of the present invention as defined by the appended claims.

1. A method for concentration of oligonucleotides from a solution comprising negatively charged oligonucleotides, the method comprising the steps of:
circulating the solution through an ultrafiltration or nanofiltration unit comprising a membrane having a nominal molecular weight cutoff in the range of from about 700 daltons to about 5000 daltons, a negatively charged surface with a zeta potential of −20 mV or lower, and a water flux in the range of 800 to 1500 ml/min/m₂;
filtering the solution through the membrane to remove salts from the solution and obtain a retentate solution and a permeate solution, wherein the oligonucleotides are retained in the retentate solution and the removed salts are contained in the permeate solution;
diafiltering the retentate solution with a diafiltration buffer to produce a concentrated oligonucleotide solution; and
collecting the concentrated oligonucleotide solution.

2. The method according to claim 1, wherein the negatively charged oligonucleotides comprise from about 5 to about 300 nucleotides.

3. The method according to claim 2, wherein the negatively charged oligonucleotides comprise from about 8 to about 50 nucleotides.

4. The method according to claim 3, wherein the negatively charged oligonucleotides comprise from about 15 to about 30 nucleotides.

5. The method according to claim 4, wherein the negatively charged oligonucleotides comprise from about 18 to about 25 nucleotides.

6. The method according to claim 1, wherein the negatively charged oligonucleotides have a zeta potential of from about −1 to −500 mV.

7. The method according to claim 1, wherein circulating of the solution through the ultrafiltration or nanofiltration unit and filtering of the solution is by tangential flow filtration.

8. The method according to claim 1, wherein prior to circulating of the solution through the ultrafiltration or nanofiltration unit, the ultrafiltration or nanofiltration unit is flushed with purified water at a cross flow rate of 2 to 10 L/min/m₂ and an average transmembrane pressure of 20 to 60 psi.

9. The method according to claim 1, wherein the solution is fed onto the membrane at a cross-flow rate of 2 to 10 L/min/m₂ and average transmembrane pressure of 20 to 60 psi.

10. The method according to claim 9, wherein the solution is fed onto the membrane at a cross-flow rate of approximately 4 to 6 L/min/m₂ and an average transmembrane pressure of approximately 35 psi.

11. The method according to claim 1, wherein an oligonucleotide
concentration of the solution is 20 to 200 OD/mL and wherein an oligonucleotide concentration of the concentrated oligonucleotide solution is up to 4,000 to 8,000 OD/mL, the concentrations being measured in Optical Density (OD) per ml by UV analysis.

12. The method according to claim 1, wherein the solution is filtered until concentrated to a volume concentration of 2.5% to 25%.

13. The method according to claim 1, wherein the method is carried out
until the conductivity of the permeate solution is from 10 to 200 micro-siemens per cm.

14. The method according to claim 1, wherein the membrane is selected
from the group consisting of a flat plate device, a flat sheet cassette, a spiral wound cartridge, a hollow fiber device, a tubular device and a single sheet device.

15. The method according to claim 14, wherein the membrane is a spiral wound cartridge.

16. The method according to claim 15, wherein the membrane is a composite semipermeable membrane comprising a polyester web, a polysulfone substrate cast on the polyester web, and a sulfonated polyether sulfone surface layer on the polysulfone substrate.

17. The method according to claim 16, wherein the sulfonated polyether sulfone surface layer has a thickness of 0.3 microns.

18. The method according to claim 16, wherein the membrane has a nominal molecular weight cutoff (MWCO) of around 720 daltons to 5 KD.

19. The method according to claim 18, wherein the nominal MWCO of the membrane is around 1 KD to 3 KD.

20. The method according to claim 16, wherein the membrane has a negative surface charge.

21. The membrane according to claim 20, wherein the sulfonated polyether sulfone surface layer of the membrane has a zeta potential of approximately −10 mV or greater negative charge.

22. The membrane according to claim 21, wherein the sulfonated polyether sulfone surface layer of the membrane has a zeta potential of approximately −20 mV or greater negative charge.

23. The membrane according to claim 22, wherein the sulfonated polyether sulfone surface layer of the membrane has a zeta potential of approximately −30 mV or greater negative charge.
